(19) United States
(12) Patent Application Publication
(10) Pub. No.: US 2025/0179152 A1
(43) Pub. Date: Jun. 5, 2025

(54) SARS-COV-2 NEUTRALIZING SYNTHETIC PROTEINS

(71) Applicant: THE TEXAS A&M UNIVERSITY SYSTEM, College Station, TX (US)

(72) Inventors: Zhilei CHEN, College Station, TX (US); Vikas CHONIRA, College Station, TX (US); Rudo SIMEON, College Station, TX (US)

(21) Appl. No.: 18/837,026

(22) PCT Filed: Feb. 10, 2023
(86) PCT No.: PCT/US2023/062350

§ 371 Date: Aug. 8, 2024

Related U.S. Application Data
(60) Provisional application No. 63/309,147, filed on Feb. 11, 2022.

(51) Int. Cl. C07K 16/10; A61K 9/00; A61K 39/00; A61P 31/14

(52) U.S. Cl. CPC C07K 16/1003; A61K 9/0043; A61P 31/14; A61K 2039/505; A61K 2039/543; A61K 2039/545; C07K 2317/76; C07K 2318/20; C07K 2319/70

(57) ABSTRACT

The present disclosure relates to compositions and methods for the treatment and/or prevention of SARS-COV-2 infections. In one embodiment the method comprises the delivery of a pharmaceutical composition comprising a SARS-COV-2 neutralizing synthetic protein, optionally wherein the protein is a trimeric protein composed of designed ankyrin repeat protein (DARPin) fused with a T4 foldon peptide that is administered non-invasively such as by nasal inhalation.

### CROSS REFERENCE TO RELATED APPLICATIONS

This application claims priority to U.S. Provisional Application 63/309,147, filed Feb. 11, 2022, the contents of which are incorporated herein by reference.

### GOVERNMENT SUPPORT CLAUSE

This invention was made with government support under DP2AI136600 awarded by National Institutes of Health. The government has certain rights in the invention.

### REFERENCE TO SEQUENCE LISTING SUBMITTED ELECTRONICALLY

Incorporated by reference in its entirety is a computer-readable nucleotide/amino acid sequence listing submitted concurrently herewith and identified as follows: as a 25 kilobytes xml file named “78090-383463.xml”, created on Feb. 9, 2023.

### BACKGROUND

Severe acute respiratory syndrome coronavirus 2 (SARS-COV-2) has infected over 350 million people worldwide resulting in over 5.6 million deaths as of February 2022. Multiple SARS-COV-2 variants with increased infectivity have emerged, which jeopardize the utility of current vaccines and therapeutic antibodies. Several monoclonal antibody (mAb) therapeutics have received emergency use authorization and demonstrated efficacy in patients. However, they suffer from high production cost, limited global supply, and inconvenient routes of administration. In addition, many human-derived antibodies show reduced efficacy against newly evolved viral variants. Accordingly, a need exists for additional therapeutic treatments for treating SARS-COV-2 infections.

In accordance with the present disclosure novel SARS-COV-2 neutralizing synthetic proteins are provided as therapeutic agents for treating SARS-COV-2 infections. The synthetic proteins comprise ankyrin repeat proteins (DARPins) engineered to mimic human ACE2 (hACE2) binding to the receptor-binding domain (RBD) of the SARS-COV-2 spike protein. DARPins represent a versatile synthetic binder scaffold that enjoys high thermostability and low immunogenicity that can ben engineered to bind a wide range of targets with pico-to nanomolar affinities.

### SUMMARY

As disclosed herein applicant has engineered two ultra-potent and broadly neutralizing synthetic proteins, named FSR16m and FSR22, for use in treating SARS-COV-2 infections, including the prevention and treatment of coronavirus disease 2019 (COVID-19) and its variants. The active domains of FSR16m and FSR22 (SR16m and SR22) are designed to specifically bind to the SARS-COV-2 spike protein. Trimerization of RBD-binding DARPins SR16m and SR22 with a T4 foldon molecule produced FSR16m and FSR22 and increased the neutralization potency of the original SR16m and SR22 proteins by 35,000- and 3,800-fold, respectively.

Remarkably, despite using the Wuhan-1 historical spike protein as the target of engineering, both FSR16m and FSR22 exhibit 30-300-fold enhanced neutralization potency against a panel of SARS-COV-2 variants of interest (VOI) and concern (VOC) including the Omicron (B.1.1.529) strain relative to the historical virus. Although the improvement in potency toward newly emerged viral variants was not intentionally built into the current design, the broad-spectrum neutralizing activity of these DARPins is likely a direct result of applicant's engineering approach which combines the selection of DARPins that mimic the viral receptor (i.e. hACE2) and the mirroring of the spike protein tertiary structure through DARPin trimerization, which enhances binding avidity. This two-pronged strategy may be useful for the engineering of protein-based therapeutics against targets that evolve over time.

The IC₅₀ values of FSR16m against authentic Beta (B.1.351) and Delta (B.1.617.2) variants were 3.4 ng/ml (47 pM) and 2.2 ng/ml (31 pM), respectively, on par with the current therapeutic antibodies. In contrast to many therapeutic antibodies that suffer from diminishing potency toward newly emerged viral variants, FSR16m and FSR22 exhibit a progressively increased neutralization activity toward these variants, culminating in the neutralization of Omicron spike pseudotyped lentivirus with IC₅₀ values of 8.5 pM and 6.2 pM, respectively, which are 35- and 296-fold more potent than that toward the lentiviruses pseudotyped with spike proteins from Wuhan-1 virus. The strong neutralization potency, combined with its broad neutralization spectrum, render proteins FSR22 and FSR16m as promising candidates as the active agent in a nasally delivered therapeutic for treating and/or preventing COVID.

Cryo-EM structures of these DARPins in complex with trimeric spike protein revealed that these DARPins recognize a region at the tip of the RBD (residues 455-456, 486-489) overlapping the ACE2-binding surface that is highly resistant to escape. Intranasally-administered FSR16m protected K18-hACE2 transgenic mice inoculated with B.1.617.2 variant with reduced weight loss and 10-100-fold reductions in viral burden in the upper and lower respiratory tract. The strong potency, broad neutralization spectrum combined with high storage stability and manufacturability render FSR16m and FSR22 promising candidates for treating and preventing infection from not only current strains of SARS-COV-2, but likely also future strains as well.

In accordance with one embodiment a pharmaceutical composition and methods for the treatment and/or prevention of SARS-COV-2 infections is provided. In one embodiment the method comprises the delivery of a pharmaceutical composition comprising a SARS-COV-2 neutralizing synthetic protein, optionally wherein the neutralizing protein is a trimeric protein composed of designed ankyrin repeat protein (DARPin) fused with T4 foldon. In one embodiment the DARPin comprises the sequence of SEQ ID NO: 5 or SEQ ID NO: 6. In one embodiment the SARS-COV-2 neutralizing synthetic protein comprises a protein having at least 95% sequence identity to SEQ ID NO: 10 or SEQ ID NO: 11. In one embodiment a pharmaceutical composition comprising a SARS-COV-2 neutralizing synthetic protein is formulated for administration using standard administration routes, optionally wherein the route of administration is non-invasive. In one embodiment the pharmaceutical composition is formulated for contact with a patient's mucosal surface, optionally via a non-invasive route. In one embodiment the pharmaceutical composition is formulated for delivery by nasal inhalation.

### BRIEF DESCRIPTION OF THE DRAWINGS

FIG. 1 shows the amino acid sequences of SARS-COV-2 neutralizing synthetic proteins FR22 (SEQ ID NO: 11), FSR16m (SEQ ID NO: 10), SR22 (SEQ ID NO: 9), SR16m (SEQ ID NO: 8) and SR16 (SEQ ID NO: 7). The T4 foldon amino acid sequence present in FR22 and FSR16m is shaded in gray.

FIGS. 2A and 2B: are graphs showing neutralization of FSR16m (FIG. 2A) and FSR22 (FIG. 2B) against lentiviruses pseudotyped with spike proteins from the wild-type and strains B.1.351 (beta) and B.1.617.2 (delta). The error bars represent standard deviation from at least two independent experiments carried in triplicates.

FIGS. 3A-3D: Intranasally administered FSR16m protects mice against infection by SARS-COV-2 B.1.617.2. FIG. 3A presents data showing weight changes in mice following infection with 10³ FFU of SARS-COV-2 (B.1.617.2) followed by no further treatment (◯) or intranasal administration of FSR16m (●). Viral RNA levels were measured at 7 days post infection (dpi) in the lung (FIG. 3B), heart (FIG. 3C) and nasal wash (FIG. 3D) for both control (◯) and mice treated with FSR16 (●). The data from FIGS. 2B-2D demonstrated the effectiveness of FSR16 to decrease viral RNA levels relative to control (n=3, 2 experiments). Two-way ANOVA with sidak's post-test, *p<0.05, ** p<0.01, comparison to the PBS-treated group.

FIG. 4: shows the amino acid residues of the SARS-COV-2 spike protein RBD (SEQ ID NO: 15) contacting FSR22 (line 1), FSR16m (line 2) and ACE2 (line 3) as indicated by highlighted residues. Amino acid residues of RBD that underwent mutations in VOCs are indicated with an asterisk. Amino acid residues that form a core footprint of monomeric and trimeric DARPins are highlighted and underlined.

FIG. 5 provides a schematic drawing of the trimeric DARPin protein structure.

FIGS. 6A & 6B present data from a FSR16m stability study. (0.85 mg/ml, 11.87 uM) was stored in PBS at room temperature. An aliquot was withdrawn each week, flash frozen and stored at −80° C. until tested at the end of the study for neutralization activity against lentivirus pseudotyped with spike protein from B.1.351 (FIG. 6A; one experiment, three technical repeats). The resulting IC₅₀ values are provided in FIG. 6B.

### DETAILED DESCRIPTION

### Definitions

In describing and claiming the invention, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

As used herein, the term “treating” includes alleviation of the symptoms associated with a specific disorder or condition and/or preventing or eliminating said symptoms.

As used herein an “effective” amount or a “therapeutically effective amount” of a therapeutic compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein, the term “conservative amino acid substitution” is defined herein as exchanges within one of the following five groups:
I. Small aliphatic, nonpolar or slightly polar residues: Ala, Ser, Thr, Pro, Gly;
II. Polar, negatively charged residues and their amides: Asp, Asn, Glu, Gln;
III. Polar, positively charged residues: His, Arg, Lys; Ornithine (Orn)
IV. Large, aliphatic, nonpolar residues: Met, Leu, Ile, Val, Cys, Norleucine (Nle), homocysteine
V. Large, aromatic residues: Phe, Tyr, Trp, acetyl phenylalanine.

As used herein the term “patient” without further designation is intended to encompass any warm blooded vertebrate domesticated animal (including for example, but not limited to livestock, horses, cats, dogs and other pets), mammals, and humans receiving a therapeutic treatment either with or without supervision by a physician.

As used herein a “designed ankyrin repeat protein” (DARPin) includes any protein comprised of ankyrin repeat peptides that has been designed and synthesized for highly specific and high-affinity binding to a target protein.

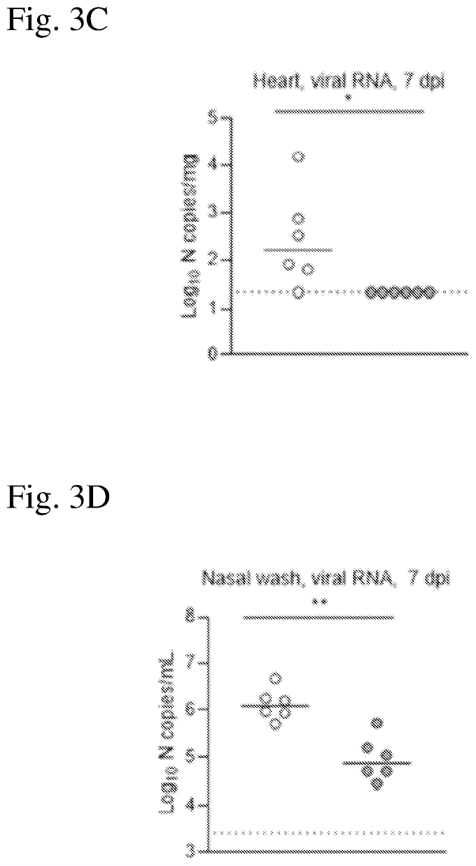

As used herein a “trimeric foldon peptide” includes any peptide that is capable of self-assembly under physiological conditions with other like peptides to form a trimeric structure held together by hydrogen and ionic bonding. Covalent linkage of compounds (e.g., proteins) to the trimeric foldon peptide will result in assembly of the linked compound into a trimeric structure. See for example FIG. 4.

### Embodiments

In accordance with one embodiment two highly potent and broadly neutralizing synthetic proteins, FSR16m and FSR22, have been created for preventing and treating SARS-CoV-2 infections, including coronavirus disease 2019 (COVID-19). The active domains of FSR16m (SEQ ID NO: 10) and FSR22 (SEQ ID NO: 11) are designed ankyrin repeat proteins (DARPins) engineered to mimic human angiotensin-converting enzyme 2 (hACE2) binding to the receptor-binding domain (RBD) of the spike protein. DARPin is a versatile synthetic binder scaffold that has high thermostability and has been engineered to bind an array of targets with pico-to nanomolar affinities, including the SARS-COV-2 spike protein. In accordance with one embodiment a SARS-COV-2 spike protein specific DARPin is provided having the sequence of SEQ ID NO: 5 or SEQ ID NO: 6.

Trimerization of RBD-binding DARPins SR16m (SEQ ID NO: 8) and SR22 (SEQ ID NO: 9) with a T4 foldon molecule (SEQ ID NO: 13) increased their neutralization potency by >300-fold. Remarkably, and despite using the Wuhan-1 historical isolate spike protein as the target of engineering, both FSR16m and FSR22 exhibit substantially enhanced neutralization potency against a panel of SARS-COV-2 variants of interest (VOI) and variants of concern (VOC) relative to the ancestral virus in pseudovirus assays. Cryo-electron microscopy (cryo-EM) studies confirmed that both SR16m and SR22 target an essential ACE2-binding epitope on the RBD, providing support for the idea that these DARPins may remain effective against future SARS-COV-2 variants. Although the improvement in neutralization potency against some newly emerged viral variants was not built into our DARPins design, the broad-spectrum activity is a direct result of our engineering approach that combines protein trimerization to increase avidity with the selection of DARPins that can compete with the viral receptor (that is, hACE2).

Several small protein domains can facilitate trimerization of proteins. The two most widely used trimerization domains in biochemical and biomedical research are the isoleucine zipper (IZ) based on the GCN4 transcriptional activator from Saccharomyces cerevisae, and the foldon domain of the bacteriophage T4 fibritin protein. The foldon domain (T4 foldon) constitutes the C-terminal 40 amino acid residues of the trimeric protein fibritin from bacteriophage T4 (SEQ ID NO: 13). In accordance with one embodiment a method of enhancing the specific binding avidity of a DARPin for its target protein is provided. The method comprises first identifying a DARPin that mimics a viral receptor used for infection of cells, and then covalently linking the identified DARPin to a peptide comprising the sequence of AYVRKDGEWVLL (SEQ ID NO: 14) or another trimeric foldon peptide. In on embodiment the identified DARPin is covalently linked to the T4 trimeric foldon peptide of SEQ ID NO: 13.

In accordance with embodiment 1, a SARS-COV-2 neutralizing protein is provided, wherein the protein comprises a designed ankyrin repeat protein (DARPin) having at least 80%, 90%, 95% or 99% sequence identity to SEQ ID NO: 12,, SEQ ID NO: 5 or SEQ ID NO: 6, optionally wherein the DARPin has the sequence of SEQ ID NO: 12, SEQ ID NO: 5 or SEQ ID NO: 6.

In accordance with embodiment 2, the SARS-COV-2 neutralizing protein of embodiment 1 is provided, wherein said neutralizing protein further comprises a trimerization domain covalently linked to said DARPin, optionally wherein the trimerization domain is a trimeric foldon peptide comprising the sequence of SEQ ID NO: 14.

In accordance with embodiment 3, the SARS-COV-2 neutralizing protein of embodiment 1 or 2 is provided, wherein said DARPin comprises an amino acid sequence of GSDLGKKLLEAARAGQDDEVRILMANGADVNAX₁DX₂X₃GX₄TPLHLAAX₅X₆GHLEIV EVLLKX-GADVNAX₇DX₉X₁₀GRTPLHLAAX₁₁X₁₂GHLEIVEVLLKX₁₃GADVNACDLX₁₄G YTPLHLAAGX₁₅GHLEIVEVLLKNGAX₁₆VNAQDKFGKTAFDISIDNGNEDLAEILQSSS (SEQ ID NO: 12), wherein
X₁-X₁₆ are independently any amino acid, optionally wherein X₁ is Cys or Leu; X₂ is Pro; X₃ is Ser; X₄ is Leu, Ile or Val; X₅ is Asp or Glu; X₆ is His, Lys or Arg; X₇ is Tyr; X₈ is Met; X₉ is Val; X₁₀ is Trp or Phe; X₁₁ is Phe; X₁₂ is Ala, Ser, Thr or Gly; X₁₃ is Tyr; X₁₄ is Tyr; X₁₅ is Arg; and X₁₆ is Gly; or
X₁ is Cys or Leu; X₂ is Leu; X₃ is Phe; X₄ is Ile or Val; X₅ is Asp or Glu; X₆ is Lys or Arg; X₇ is Asn; X₈ is Gly; X₉ is Ala; X₁₀ is Trp or Phe; X₁₁ is Leu; X₁₂ is Thr or Gly; X₁₃ is Asn; X₁₄ is Asn; X₁₅ is Leu and X₁₆ is Asp.

In accordance with embodiment 4, the SARS-COV-2 neutralizing protein of any one of embodiments 1-3 is provided, wherein said trimeric foldon peptide comprises the sequence of AYVRKDGEWVLL (SEQ ID NO: 14).

In accordance with embodiment 5, the SARS-COV-2 neutralizing protein of any one of embodiments 1˜4 is provided, wherein said DARPin comprises an amino acid sequence selected from the group consisting of SEQ ID NO: 5, SEQ ID NO: 6, SEQ ID NO: 7, SEQ ID NO: 8 and SEQ ID NO: 9.

In accordance with embodiment 6, the SARS-COV-2 neutralizing protein of any one of embodiments 1-5 is provided further comprising a sequence of SEQ ID NO: 13 covalently linked to said protein.

In accordance with embodiment 7, the SARS-COV-2 neutralizing protein of any one of embodiments 1-6 is provided, wherein said neutralizing protein comprises an amino acid sequence having at least 80%, 90%, 95% or 99% sequence identity with SEQ ID NO: 10 or SEQ ID NO: 11.

In accordance with embodiment 8, a pharmaceutical composition is provided comprising a SARS-COV-2 neutralizing protein of any one of embodiments 1-7 and a pharmaceutically acceptable carrier.

In accordance with embodiment 9, the pharmaceutical composition of embodiment 8 is provided wherein the composition is formulated for intranasal delivery.

In accordance with embodiment 10, a method of enhancing the specific binding avidity of a DARPin protein for its target protein is provided, wherein the method comprising covalently linking the DARPin protein to a trimeric foldon peptide, optionally wherein the DARPin protein specifically binds to a viral capsid protein, optionally wherein the DARPin protein specifically binds to a SARS-COV-2 virus spike protein, optionally wherein the DARPin protein is selected from SEQ ID NO: 5 or SEQ ID NO: 6, optionally wherein the trimeric foldon peptide comprises the sequence of AYVRKDGEWVLL (SEQ ID NO: 14), or the sequence of SEQ ID NO: 13.

In accordance with embodiment 11, a method of treating a patient exposed to a SARS-COV-2 virus is provided, wherein the method comprises the administration of a pharmaceutical composition of any one of claims 1-7.

In accordance with embodiment 12, the method of treating a patient according to embodiment 11 is provided, wherein the patient has been identified as having a SARS-COV-2 infection.

In accordance with embodiment 13, the method of treating a patient according to embodiment 11 or 12 is provided, wherein the patient is administered a pharmaceutical composition comprising a SARS-COV-2 neutralizing protein of any one of embodiments 1-7 within 1, 2, 6, 12 hours, or within 1, 2, 3, 4, 5, 6, 7, 8, 9, 10, 11, 12, 13, 14 days after appearance of symptoms of infection or a positive test for infection.

In accordance with embodiment 14, the method of treating a patient according to any one of embodiments 11-13 is provided wherein the method comprises the administration of an amino acid sequence having 95% sequence identity with SEQ ID NO: 10 or SEQ ID NO: 11, optionally wherein said amino acids sequence comprises SEQ ID NO: 10 or SEQ ID NO: 11.

In accordance with embodiment 15, the method of treating a patient according to any one of embodiments 11-14 is provided wherein the method comprises the administration of one or more SARS-COV-2 neutralizing proteins, selected from the group consisting of sequence SEQ ID NO: 10 and SEQ ID NO: 11, optionally when two SARS-COV-2 neutralizing proteins are administered, they are administered sequentially within 2, 4, 8, 12, 24, 48 or 72 hours of each other. In an alternative embodiment when two SARS-COV-2 neutralizing proteins are administered, they are administered simultaneously.

In accordance with embodiment 16, the method of treating a patient according to any one of embodiments 11-15 is provided wherein the pharmaceutical composition is administered intranasally.

In accordance with one embodiment suitable DARPins for use in the present invention can be identified by screening libraries of potential candidates for binding to the predetermined target. For example, employing an in-house DARPins library with greater than 109 distinct clones displayed on M13 phage particles, applicant sequentially enriched binders to biotinylated RBD and the full-length spike protein (Wuhan-1 strain) over four rounds of phage panning (See Example 1). The enriched DARPin library pool from the final round was cloned into an expression vector with 6×his and Myc tags at the N-terminus and transformed into BL21(DE3) Escherichia coli cells. Three hundred colonies were picked and grown in 96-well plates, and the cell lysate was subjected to enzyme-linked immunosorbent assay (ELISA)-based screens. Eleven unique clones bound strongly to both the RBD and the full-length spike protein and were purified by nickel-affinity chromatography. A competition ELISA was used to identify DARPin molecules that could inhibit binding between hACE2 and spike proteins and yielded two unique clones: SR16 (SEQ ID NO: 7) and SR22 (SEQ ID NO: 9). Sodium dodecyl sulfate-polyacrylamide gel electrophoresis (SDS-PAGE) analysis of these proteins revealed a homogenous band for SR22 and an additional band for SR16. The N-terminal 6×his tag in SR16 was moved to the C-terminus to give rise to SR16m (SEQ ID NO: 8).

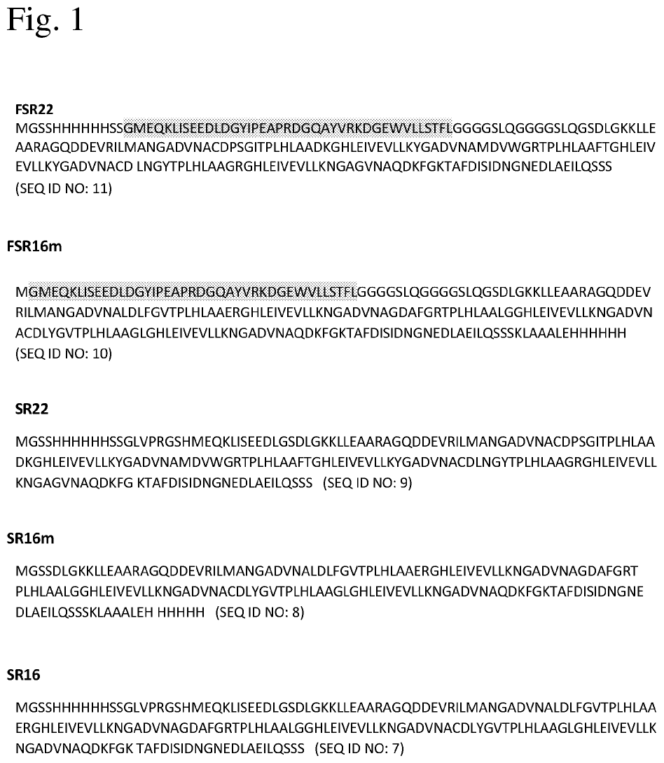

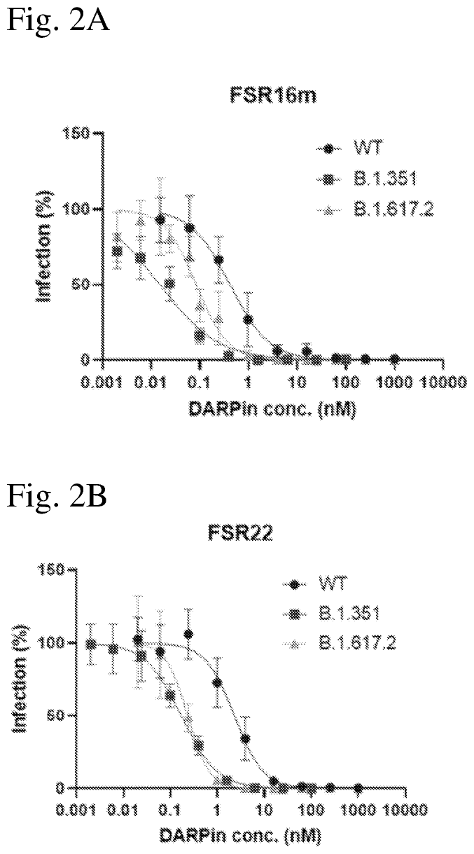

Because the SARS-COV-2 spike protein adopts a trimeric structure, applicant trimerized both SR16m and SR22 through fusion to a small trimeric protein, T4 foldon (SEQ ID NO: 13), via a flexible (GGGGSLQ; SEQ ID NO: 1)×2 linker to form FSR16m (SEQ ID NO: 10 and FSR22 (SEQ ID NO: 11, respectively; See FIG. 1). These trimeric DARPins were discovered to bind the spike protein with much higher avidity. Subsequently, using lentiviruses pseudotyped with the Wuhan-1 spike protein, we determined the neutralization activity of SR16m and SR22 (FIGS. 2A and 2B). Monomeric SR16m and SR22 showed weak inhibitory activity against the historical Wuhan-1 virus with 50% inhibitory concentration (IC₅₀) values of 1 and 0.7 μm, respectively, which were dramatically enhanced upon trimerization (Table 1).

TABLE 1
Summary of the Neutralization IC₅₀ Values for pseudotyped lentiviruses

| | | | B.1.351 | | B.1.617.2 | | C.37 | | B.1.1.529 | |
|---|---|---|---|---|---|---|---|---|---|---|
| IC₅₀ | WT | | (beta) | | (delta) | | (lambda) | | (omnicron) | |
| SR16m | 11.6 | μM | 0.42 | μM | 77.8 | nM | N.D. | | N.D. | |
| SR22 | 0.7 | μM | 1.1 | μM | 69.8 | nM | N.D. | | N.D. | |
| FSR16m | 23.7 | ng/ml | 1.3 | ng/ml | 4.8 | ng/mL | 0.9 | ng/ml | 0.6 | ng/ml |
| | 331 | pM | 18 | pM | 66 | pM | 13 | pM | 9. | SpM |
| FSR22 | 130 | ng/ml | 9.5 | ng/ml | 13.3 | ng/ml | 6.3 | ng/ml | 0.4 | ng/ml |
| | 1834 | pM | 13 | SpM | 188 | pM | 89 | pM | 6.1 | pM |

Despite the use of the Wuhan-1 spike protein during DARPin engineering, both FSR16m and FSR22 exhibited greater neutralization activity toward viruses pseudotyped with spike proteins from VOCS. As an example, the IC₅₀ values of FSR16m and FSR22 against pseudoviruses displaying the RBD from the Omicron variant are 8.5 and 7.3 pm, respectively, which are substantially more potent than toward viruses displaying Wuhan-1 spike protein (331 pm and 1.8 nm, respectively).

All monomeric and trimeric DARPin proteins were efficiently expressed in E. coli and easily purified by one-step immobilized metal affinity chromatography. The presence of surface Cys residues did not impact the tertiary structure of these proteins. Monomeric DARPin SR16m migrated slightly faster than SR22 on SDS-PAGE gel and eluted later on size exclusion chromatography, while their respective trimers had nearly identical size. FSR16m was homogenous in solution, whereas a small percentage of FSR22 appeared to aggregate under nonreducing conditions.

FSR16m and FSR22 bind diverse RBD variants with high avidity. FSR16m exhibited superior binding avidity relative to FSR22 against a panel of spike proteins from VOC and VOI as measured in a biolayer interferometry binding assay (see Table 2).

TABLE 2
Summary of the 50% maximum effective binding concentrations
(EC₅₀) to RBD proteins with indicated mutations. (ELISA EC₅₀ nM)

| | viral strain | FSR16m | FSR22 |
|---|---|---|---|
| Natural variant | Wuhan-1 | 0.59 | 1.96 |
| Natural variant | K417N + E484K + N501Y | 0.43 | 3.79 |
| Natural variant | E484K + N501Y | 0.23 | 2.68 |
| Natural variant | N439K | 0.75 | 2.63 |
| Natural variant | L452R | 0.37 | 1.87 |
| Natural variant | N501Y | 0.24 | 1.66 |
| Natural variant | L452R + E484Q | 0.21 | 4.05 |
| Natural variant | L452R + T478K | 0.13 | 7.83 |
| Natural variant | S477N | 0.63 | 1.57 |
| Natural variant | T478K | 0.19 | 15.18 |
| Natural variant | E484Q | 0.34 | 4.05 |
| Natural variant | F486S | 300.00 | 70.54 |
| LY-CoV55-resistant | E484K | 0.21 | 4.57 |
| LY-CoV55-resistant | F490S | 0.15 | 1.57 |
| LY-CoV55-resistant | Q493R | 0.75 | 1.24 |
| LY-CoV55-resistant | S494P | 0.26 | 0.61 |
| REGN-10933-resistant | K417E | 300.00 | 300.00 |
| REGN-10933-resistant | Y453F | 0.74 | 3.97 |
| REGN-10933-resistant | L455F | 0.10 | 9.24 |
| REGN-10933-resistant | G476S | 5.73 | 27.19 |
| REGN-10933-resistant | F486V | 300.00 | 104.70 |
| REGN-10933-resistant | Q493K | 0.49 | 2.42 |
| REGN-10987-resistant | K444Q | 1.03 | 5.92 |
| REGN-10987-resistant | V445A | 0.46 | 3.43 |
| REGN-10987-resistant | G446V | 7.65 | 30.60 |

Due to the negligible dissociation rate, the KDapp values of FSR16m for the RBD of Wuhan-1 and six viral variants were below the detection limit (KDapp<1 pM). In the case in pseudovirus assays of Omicron RBD, a faster dissociation rate was observed, resulting in KDapp of 3.65 nM. This result contrasted with our neutralization assay in which the FSR16m exhibited improved IC₅₀ values against lentiviruses displaying RBD from the Omicron variant compared to Wuhan-1 virus. The difference in the apparent binding avidity may be due to differences in the tertiary conformation of the protein in solution and on the virus. The binding study used dimeric Fc-tagged RBD. FSR22 maintained a similar binding avidity toward all tested RBDs with a slight increase in avidity to the RBD of Omicron (KDapp=2.63 nM) compared to Wuhan-1 (KDapp=12.3 nM). Overall, a faster dissociation rate was observed for FSR22 than FSR16m.

To assess the breadth of FSR16m and FSR22 interaction with other SARS-COV-2 VOC and VOI, we determined their binding to a panel of 24 RBD mutants by ELISA. Remarkably, FSR16m maintained nanomolar EC50 values toward all variants except for the ones with K417E and F486V/F486S substitutions (Table 2). While the K417E variant was predicted to escape antibody neutralization based on the pseudotyped virus studies, this amino acid change also resulted in reduced binding affinity of spike for hACE2 (17.8% of Wuhan-1 control) due to the loss of a critical salt bridge interaction between the positively charged K417 in the spike protein and the negatively charged Asp in hACE2. K417N/K417T substitutions are present in B.1.351 (β) and P.1 (γ) variants. The affinity of FSR16m for the B.1.351 RBD (K417N+E484K+N501Y, EC50 0.43 nM) is similar to Wuhan-1 RBD (EC50 of 0.59 nM) but slightly weaker than that for a variant with only E484K+N501Y (EC50 of 0.23 nM), indicating that the K417N mutant weakens the interaction slightly between FSR16m and the spike protein. Similarly, the variants F486V and F486S also exhibit decreased hACE2-binding affinity (37% and 57%, respectively, of Wuhan-1 control). Consequently, variants with these mutations (for example, BA.4/.5) may exhibit reduced viral fitness and virulence. Mutations G476S and G446V enable resistance to neutralization by antibodies REGN-10933 and REGN-10987, respectively. Nonetheless, and despite a greater than 10-fold increase in EC50 values, FSR16m still maintained nM binding avidity to spike proteins with both of these variants (Table 2). However, no detectable interaction was observed between FSR16m and the RBD proteins of sarbecoviruses of clade 1a (SARS-COV-1, RaTG13, WIV1), clade 2 (BtkY72) and clade 3 (Rs4081) using the same binding assay, indicating that the epitope of FSR16m is not conserved among other sarbecoviruses.

FSR22 retained high-avidity binding to most of the tested variants with EC50<10 nM although its binding strength was generally lower than FSR16m. Similar to FSR16m, FSR22 also exhibits lower avidity to RBDs harboring variants K417E, F486V/F486S, G446V or G476S. Beyond these, FSR22 had reduced avidity to RBDs containing a T478K substitution, pointing to its engagement of a slightly different binding interface than FSR16m. The ability of FSR16m and FSR22 to bind diverse RBD variants with high avidity may stem from our engineering approach, which aimed to identify binders that mimic hACE2 engagement, a step that the virus is obligated to maintain as high-affinity binding for efficient infection.

The capacity of FSR16m and FSR22 to neutralize authentic SARS-COV-2 in Vero-hACE2-TMPRSS2 cells was tested. Both FSR16m and FSR22 DARPins efficiently inhibited infection of several authentic SARS-COV-2 strains (Tables 3).

TABLE 3
Summary of the Neutralization IC₅₀ Values against authentic SARS-CoV-2 viruses

| IC₅₀ | | | B.1.617.2/ | B.1.1.519/ | B.1.1.519/ | B.1.1.519/ |
|---|---|---|---|---|---|---|
| (ngml⁻¹) | B.1.351 | B.1.617.2 | AY1 | BA.1 | BA.1.1 | BA.2 |
| FSR16m | 3.4 | 2.2 | 3.3 | 44.7 | 7.4 | 33.3 |
| FSR22 | 57.2 | 41.7 | 44.2 | 169.2 | 41.2 | 216.2 |

The IC₅₀ values of FSR16m against B.1.351, B.1.617.2 and B.1.617.2.AY1 viruses were 3.4, 2.2 and 3.3 ng/ml, respectively, values comparable to that of the Regen-COV antibody cocktail. While both DARPins neutralized authentic Omicron strains, FSR16m was more potent. The IC₅₀ values of FSR16m and FSR22 against BA.1, BA.1.1 and BA.2 strains were 44.7 and 169.2 ng/ml, 7.4 and 41.2 ng/ml, and 33.3 and 216.2 ng/ml, respectively (Table 3).

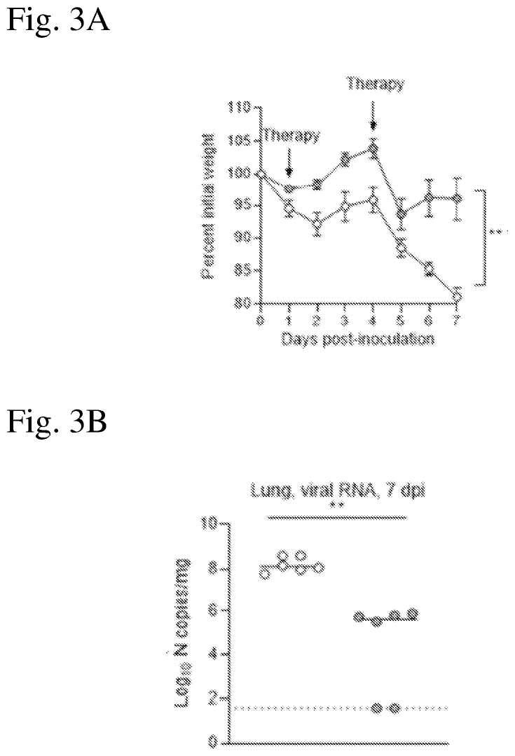

Given the potency of FSR16m, we investigated the in vivo efficacy of this DARPin in mice. Eight-week-old female heterozygous K18-hACE2 C57BL/6J13 mice were administered 10³ focus-forming units (FFU) of SARS-COV-2 B.1.617.2 on day 0. This dose of SARS-COV-2 was determined to cause severe lung infection and inflammation in previous studies. On days 1 and 4 post infection, FSR16m (50 μg per mouse in PBS) was administered via an intranasal route. FSR16m-treated mice had less weight loss and 10- to 100-fold lower levels of viral RNA in the lung, heart and nasal wash than mice treated with PBS (FIGS. 3A-3D). Mice treated with FSR16m also showed lower levels of several proinflammatory cytokines compared to the control-treated, infected mice including IFN-γ (p=0.0043), IL-15 (p=0.0411), CXCL10 (p=0.0022), LIF (p=0.0411), CCL2 (p=0.0087) and CCL4 (p=0.0152). Thus, FSR16m showed post exposure therapeutic efficacy against SARS-COV-2 as an intranasally administered protein.

To understand the broadly neutralizing activity of FSR22 and FSR16m against SAR-COV-2 variants, we determined their cryo-EM structures in complex with SARS-COV-2 S 6P spike proteins. Ab initio reconstructions of cryo-EM images of the complexes followed by heterogeneous refinements revealed trimeric SARS-COV-2 spikes with a variety of RBD conformations, ranging from all RBD-down to one, two or three RBD-up conformations—with SR22 or SR16m binding to the RBD-up forms in all cases. As FSR22 and FSR16m, the trimeric forms of three SR22 or SR16m linked by a foldon were the most inhibitory, we focused on refining the three RBD-up conformations of SARS-COV-2 spikes with the FSR22 (or FSR16m) bound to the tip of the RBD in its open conformation. While the trimeric spike complexes were generally well-defined, the tip of the RBD along with FSR22 (or FSR16m) showed substantial conformational heterogeneity. As a result, we could not delineate the FSR22 (or FSR16m)-RBD interface in atomic detail, even after extensive heterogeneous refinement followed by local refinement. The overall fold of the DARPins and the tip of the RBDs did, however, fit well, and these revealed both FSR22 and FSR16m to bind at the ‘tip’ of the RBD in their ‘RBD-up’ conformation.

Although the FSR22 structure in complex with SARS-COV-2 spike was determined at slightly higher resolution than the FSR16m structure, the conformational mobility of these structures reduced their resolution. To corroborate our findings, we determined structures of these DARPin molecules in complex with RBD by itself. To provide sufficient mass for cryo-EM structure determination, we added the antigen-binding fragments (Fabs) from two antibodies (S309 and CR3022) that neither compete with each other nor with either DARPin molecule. We obtained a structure of the quaternary complex with SR22 at 4.11 Å resolution, with density for the DARPin of similar quality as RBD and Fab variable domains; we also obtained a structure of the quaternary complex with SR16m at 4.26 Å resolution, where the slightly reduced resolution related to the propensity of SR16m to self-aggregate. Despite differences in trimerization (with either spike/RBD or trimeric/monomeric DARPin), the overall fold of the two DARPins and their binding mode to SARS-COV-2 were nearly identical. The four structures suggested that the DARPins recognize a subset of RBD surface composed of residues F456, A475, F486, N487 and Y489, which overlap closely with the hACE2-binding surface.

Notably, most of these residues did not overlap with residues that changed in VOC, including N501Y in B.1.1.7 (Alpha), K417N, E484K and N501Y in B.1.351 (Beta), L452R and E484Q in B.1.617.2 (Gamma), and K417N, S477N, Q498R and N501Y in B.1.1.529 (Omicron) (FIG. 4), suggesting that the surface recognized by FSR22 (or FSR16m) is important for ACE2 binding, and changes in these residues may compromise viral infectivity and fitness. Comparison of the two DARPin-RBD complexes and the ACE2-RBD structure revealed a steric clash between a monomer of SR22 (or SR16m) and ACE2 bound to RBD, as the DARPins and ACE2 recognize overlapping binding surfaces. However, SR22 and SR16m only buried approximately 500 Å2 of surface area on RBD, whereas ACE2 binding buried approximately 970 Å2 of the surface on RBD, explaining the structural basis for the relatively poor neutralizing ability of SR22 and SR16m (monomers) as they could be outcompeted by ACE2 for RBD binding. Conversely, FSR22 and FSR16m, composed of three SR22 and SR16m, respectively, can overcome the lower affinity of monomeric DARPins to RBD by binding three RBDs in their open conformation. Collectively, the cryo-EM structures of FSR22 and FSR16m-bound SARS-COV-2 spike revealed that FSR22 and FSR16m potently neutralize SARS-COV-2 including VOCs by targeting a minimal but essential subset of ACE2-binding residues and taking advantage of avidity gained by trimerization.

### Example 1: Engineering of FSR16m and FSR22

### Methods

### Selection and Screening of Spike Protein-Binding DARPin Molecules

An in-house N3C DARPin library with greater than 109 diversity was used in the phage panning step. Purified full length spike protein (BEI 52724) and RBD (BEI52306) were biotinylated via EZ-link-sulfo-NHS-LC-biotin (Thermo Fisher, Cat #21335) and used as the target proteins. RBD was used as the target protein in round 1, 2 and 4 while the full-length spike protein was used as the target in round 3 to ensure the enrichment of DARPins able to recognize RBD present on the full-length spike protein. Round 1 used the target protein (100 nM) in solution while round 2-4 employed decreasing concentrations of the target protein immobilized on streptavidin-coated ELISA plate (100 nM, 50 nM and 20 nM). The enrichment of RBD binding DARPins was confirmed by phage ELISA against both RBD and full-length spike protein.

The enriched DARPin pool from the 4th round was cloned into the pET28a vector for high-level DARPin expression. The resulting DARPin contains a Myc tag at the N-terminus and a 6×His tag at the C-terminus. After transformation, a total of 300 individual E. coli BL21 (DE3) clones were picked and grown in deep 96-well plates (1 mL/well) at 37° C. in LB and induced with IPTG (0.5 mM) The next day cell pellets were harvested, resuspended in 200 μL PBS (1.8 mM KH₂PO₄, 10 mM Na₂HPO₄, 137 mM NaCl, 2.7 mM KCl, pH 7.4) supplemented with lysosome (200 g/mL, Amresco Cat #0663-5G). ELISA was used to identify the target binding DARPins. Briefly, Nunc MaxiSorp plates (Fisher scientific Cat #50-712-278) were first coated with 4 μg/mL neutravidin (Thermoscientific, Cat #31000) in PBS at room temperature for 2 hours. The wells were thoroughly washed with PBS and then blocked with PBS supplemented with 0.5% BSA (Fisher scientific Cat #BP9706100, PBS-B) at 4° C. overnight. The next day, after thorough washing with PBS-T (PBS with 0.1% Tween 20), the wells were incubated with biotinylated RBD or full-length spike protein (20 nM in PBS) at room temperature for 1 hours. The wells were washed again with PBS-T prior to the addition of 100 μL cell lysate (5-fold diluted in PBS) and incubated at room temperature for 2 hours. The amount of plate bound DARPin in each well was quantified using mouse anti-myc antibody (Invitrogen, Cat #13-2500, 1:2500 diluted in PBS-B) and HRP conjugated goat anti-mouse antibody (Jackson ImmunoResearch catalog #115-035-146, 1:1000 diluted in PBS-B) as the primary and secondary antibody, respectively, and BioFx TMB (VWR, Cat #100359-154) for color development. 20 and 23 of clones showed significant ability to bind to RBD and full-length spike protein, respectively. Sequencing revealed 11 unique clones with significant ability to bind both RBD and full-length spike protein.

To identify DARPin clones able to block spike protein and hACE2 interaction, a competitive ELISA was used. Briefly, Maxisorp plates were first coated with full-length spike protein (BEI 52308, 2 nM in PBS) at room temperature for 2 hours. The wells were blocked with PBS-B at 4° C. overnight, washed with PBS-T, and then incubated with mixtures of Ace2-HRP (prepared in-house, 0.5 nM) and different IMAC-purified DARPins (50 mM) 43,45 at room temperature for 1 hour. The amount of Ace2-HRP in each well was quantified using BioFx TMB.

For preparation of hACE2-HRP, hACE2 (Raybiotech, Cat #230-30165) was first biotinylated as described above and then incubated with an equal molar amount of streptavidin-HRP (JIR Cat #016-030-084) in PBS at room temperature for 20 mins. The mixture was diluted 100-fold in PBS-B or stored at −20° C. in 50% glycerol until use.

### Plasmids and Cells

Plasmids encoding the wild type Δ19 spike protein were obtained from Addgene (Cat #145780). The plasmids for the 419 spike protein of B.1.617.2 and C.37 were generously provided by Prof. Nathaniel Landau. DNA fragment encoding the 419 spike protein of strain B. 1.351, and the RBD (residues 339-501) of B.1.1.529 was synthesized by Gene Universal and inserted into the pCG1 plasmid. Prof. Paul Bieniasz provided the 293T cell clone 22 (293T.c22) with high expression efficiency of human ACE2 and the lentiviral reporter plasmid pHIV-1NL4-3-ΔEnv-NanoLuc48. The plasmid encoding the chimeric B.1.1.529 spike protein (B.1.1.529*) was constructed by replacing the RBD region (residues 339-501) in B.1.351 with that from B.1.1.529.

To construct trimeric DARPin molecules, the DNA fragment encoding a codon optimized T4 foldon (pdb:1rfo) was synthesized by Gene Universal. T4 foldon was fused to the N-terminus of a DARPin molecule via a flexible (GGGGSLQ; SEQ ID NO: 1)×2 linker and cloned into the pET28a expression vector. DARPin SR16, SR22, FSR16 and FSR22 contain a 6×His tag and a Myc tag at the N-terminus, while SR16m and FSR16m contains only a 6×His tag at the C-terminus.

### SARS-COV-2 Spike Lentiviral Pseudotypes

Lentiviral pseudotypes with various SARS-COV-2 spike proteins (CoV2pp) were produced. Briefly, plasmids encoding the Δ19 spike protein and reporter pHIV-1NL4-3-ΔEnv-NanoLuc48 (1:3 molar ratio, 10 μg total) were thoroughly mixed with 500 μL serum-free DMEM medium and 44 μL PEI (1 mg/mL, Polysciences transporter 5, Cat #26008-5) and used to transfect 5×106 293T cells seeded the night before. 24 hours post transfection, the medium was replaced with fresh DMEM supplemented with 10% FBS and 48 hours post transfection the viral supernatant was harvested, aliquoted and stored at −80° C. until use.

To determine the neutralization efficiency, serially diluted DARPin molecules were incubated with CoV2pp (final 500-fold diluted) at 37° C. for 30 minutes before being added to 293T.c22 cells seeded the night before at 10⁴ cells/well in 96 well plates. The plates were incubated at 37° C./5% CO2 for 48 hours and the NanoLuc signal from each well was quantified using the Nano-Glo Luciferase Assay kit (Promega Cat #N1120).

### DARPin Protein Production and Characterization

All DARPin molecules were expressed in E. coli B121 (DE3) cells in LB medium supplemented with 50 g/mL kanamycin. Protein expression was induced with IPTG (0.5 mM) when the culture reached OD600˜0.5. The protein expression was continued at 37° C. for 5 hours and the cells were harvested by centrifugation. The proteins were purified using gravity Ni-NTA agarose columns following the standard protocol. Protein purity was accessed using 12% SDS-PAGE gels.

For in vivo studies, the IMAC purified FSR16m was sterilized by filtration through a 0.22 μm filter, concentrated and buffer exchanged into PBS via ultrafiltration (Amicon column MWCO 10 KDa, Cat #UFC801024) before endotoxin removal using High Capacity Endotoxin Removal Spin Columns (Pierce Cat #88274). Endotoxin level in the protein sample (1.5 mg/mL) was quantified to be <30 U/mL using Pierce Chromogenic Endotoxin Quant Kit (ThermoFisher Cat #A39552).

For size exclusion chromatography studies, DARPin samples (0.9 mg/mL×0.25 mL) were loaded onto an Enrich SEC 70×300 Column equilibrated with PBS (GE AKTApure). Vitamin B12 (Sigma V2876-100 MG) was used at a final concentration of 1 mg/ml as an internal control.

### Spike RBD and DARPin Affinity Measurement

The human IgG1 Fc-tagged RBD proteins were made in-house. The affinity measurement was performed on the ForteBio Octet RED 96 system (Sartorius, Goettingen, Germany). Briefly, the RBD proteins (20 μg/ml) were captured onto protein A biosensors for 300s. The loaded biosensors were then dipped into the kinetics buffer for 10 s for adjustment of baselines. Subsequently, the biosensors were dipped into serially diluted (from 0.13 to 300 nM) DARPin proteins for 200 s to record association kinetics and then dipped into kinetics buffer for 400 s to record dissociation kinetics. Kinetic buffer without DARPin was used to correct the background. The Octet Data Acquisition 9.0 software was used to collect affinity data. For fitting of KD values, Octet Data Analysis software V11.1 was used to fit the curve by a 1:1 binding model using the global fitting method.

### ELISA Binding Assay

ELISA plates were coated with recombinant DARPin protein (2 μg/ml) at 4° C. overnight and blocked with 5% skimmed milk at 37° C. for 2 h. 100 μL serially diluted human IgG1 Fc-tagged RBD proteins in 1% skimmed milk was added to each well and the plates were incubated at room temperature for 3 h before the addition of 100 L/well HRP-conjugated goat anti-human IgG antibody (Jackson ImmunoResearch, 109-035-088, diluted 1:5,000) and the plates were incubated at room temperature for another hour. The plates were thoroughly washed (3-5 times) with PBST (0.05% Tween-20) between incubation steps. TMB (3,3′,5,5′-tetramethylbenzidine) substrate was added at 100 μl per well for colour development. The reaction was stopped by adding 50 μl per well 2M H₂SO₄. The OD450 nm was read by a SpectraMax microplate reader and analysed with GraphPad Prism 8.

### Cells and Authentic Viruses

Vero-hACE2-TMPRSS2 (a gift of A. Creanga and B. Graham, NIH) were cultured at 37° C. in Dulbecco's Modified Eagle medium (DMEM) supplemented with 10% fetal bovine serum (FBS), 10 mM HEPES pH 7.3, 1 mM sodium pyruvate, 1× non-essential amino acids, 100 U/ml of penicillin-streptomycin, and 5 μg/mL of puromycin. The B.1.617.2, B.1.617.2.AY1, and B.1.351 SARS-COV-2 variant strains were obtained from infected individuals. Infectious stocks were propagated by inoculating Vero-hACE2-TMPRSS2 cells. Supernatant was collected, aliquoted, and stored at −80° C. All work with infectious SARS-CoV-2 was performed in Institutional Biosafety Committee-approved BSL3 and A-BSL3 facilities at Washington University School of Medicine using positive pressure air respirators and protective equipment. All virus stocks were deep-sequenced after RNA extraction to confirm the presence of the anticipated substitutions.

### Mouse Experiments

Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01). Virus inoculations were performed under anesthesia that was induced and maintained with ketamine hydrochloride and xylazine, and all efforts were made to minimize animal suffering.

Heterozygous K18-hACE C57BL/6J mice (strain: 2B6.Cg-Tg (K18-ACE2) 2Prlmn/J) were obtained from The Jackson Laboratory. Animals were housed in groups and fed standard chow diets. 8-week-old female mice were administered 10³ FFU of SARS-COV-2 via intranasal administration.

### Measurement of Viral Burden

Tissues were weighed and homogenized with zirconia beads in a MagNA Lyser instrument (Roche Life Science) in 1,000 μL of DMEM media supplemented with 2% heat-inactivated FBS. Tissue homogenates were clarified by centrifugation at 10,000 rpm for 5 min and stored at −80° C. RNA was extracted using the MagMax mirVana Total RNA isolation kit (Thermo Scientific) on a Kingfisher Flex extraction robot (Thermo Scientific). RNA was reverse transcribed and amplified using the TaqMan RNA-to-CT 1-Step Kit (ThermoFisher). Reverse transcription was carried out at 48° C. for 15 min followed by 2 min at 95° C. Amplification was accomplished over 50 cycles as follows: 95° C. for 15 s and 60° C. for 1 min. Copies of SARS-COV-2 N gene RNA in samples were determined using a previously published assay 12,50. Briefly, a TaqMan assay was designed to target a highly conserved region of the N gene (Forward primer: ATGCTGCAATCGTGCTACAA, SEQ ID NO: 2; Reverse primer: GACTGCCGCCTCTGCTC, SEQ ID NO: 3; Probe:/56-FAM/TCAAGGAAC/ZEN/AACATTGCCAA/3IABKFQ/; SEQ ID NO: 4). This region was included in an RNA standard to allow for copy number determination down to 10 copies per reaction. The reaction mixture contained final concentrations of primers and probe of 500 and 100 nM, respectively.

### Authentic Virus Neutralization Assay

Serial dilutions of DARPins were incubated with 102 focus-forming units (FFU) of the indicated SARS-COV-2 strains for 1 h at 37° C. DARPin-virus complexes were added to Vero-hACE2-TMPRSS2 cell monolayers in 96-well plates and incubated at 37° C. for 1 h. Subsequently, cells were overlaid with 1% (w/v) methylcellulose in MEM supplemented with 2% FBS. Plates were harvested 24 h later by removing overlays and fixed with 4% PFA in PBS for 20 min at room temperature. Plates were washed and sequentially incubated with an oligoclonal pool of SARS2-2, SARS2-11, SARS2-16, SARS2-31, SARS2-38, SARS2-57, and SARS2-71 anti-spike protein antibodies (Vanblargan, L. et al. Cold Spring Harbor Laboratory, 2021), and HRP-conjugated goat anti-mouse IgG in PBS supplemented with 0.1% saponin and 0.1% bovine serum albumin. SARS-COV-2-infected cell foci were visualized using TrueBlue peroxidase substrate (KPL) and quantitated on an ImmunoSpot microanalyzer (Cellular Technologies). Data were processed using Prism software (GraphPad Prism 8.0).

### Cryo-EM Sample and Grid Preparation

SARS-COV-2 S6P spike protein was produced in 293F cells and purified from cell culture supernatant using a Ni-NTA agarose column followed by Superdex S200 16/600 (GE Healthcare) size-exclusion column chromatography. FSR16m and FSR22 were produced in E. coli B121 (DE3) cells in LB medium. To make SARS-COV-2 S6P and FSR16m (or FSR22) complexes, SARS-COV-2 spike and FSR16m (or FSR22) were incubated in 1:3 molar ratio and the complexes were purified by Superdex S200 GL 10/300 (GE Healthcare) using 10 mM HEPES, 7.4, 150 mM NaCl as running buffer and were confirmed by SDS-PAGE and negative stain EM. 2.3 μl of the complex at 0.5 mg/ml concentration was deposited on a C-flat grid (protochip.com). The grids were vitrified using an FEI Vitrobot Mark IV (Themo Fisher Scientific) with a wait time of 30 s, blot times of 1.5-4.5 s and blot force of 1.

### Cryo-EM Data Collection and Processing

The FSR22/16m: SARS-COV-2 spike complexes grids were imaged using a Titan Krios electron microscope equipped with a Gatan K2 Summit direct detection device. Movies were collected at 105,000× magnification over a defocus range of −1.0 μm to −2.5 μm for a 10 s with the total dose of 58.06 e-/Å2 fractionated over 50 raw frames. All data processing was done with cryoSPRACv3.3.1. Motion correction and CTF estimation in patch mode, blob particle picking, and particle extraction with the box size of 500 Å were performed followed by 2D classifications, ab initio 3D reconstruction, and multiple rounds of 3D heterogeneous refinement. C3 symmetry was applied for the final reconstruction of the FSR22/16m: SARS-CoV-2 spike complex after the initial 3D heterogeneous refinement using C1 symmetry identified a trimer with 3 RBD-up conformation bound three SR22/16m molecules. To define RBD-FSR22 interface, local refinement was performed using a soft mask covering one SR22 and one RBD molecule.

### Model Building and Refinement

Coordinates from PDB ID: 7BNO and the initial models of SR22/16m generated by using AlphaFold were used for initial fit to the reconstructed maps. Then the models were manually built using Coot (Acta crystallographica (2004) Section D, Biological crystallography 60, 2126-2132, doi: 10.1107/s0907444904019158), followed by simulated annealing and real space refinement in Phenix (Acta crystallographica (2010) Section D, Biological crystallography 66, 213-221, doi: 10.1107/S0907444909052925 iteratively. Geometry and map fitting were evaluated throughout the process using Molprobity and EMRinger. Figures were generated using PyMOL (www.pymol.org) and UCSF ChimeraX.v1.1.160.

Employing an in-house DARPin library with >109 distinct clones displayed on M13 phage particles, we sequentially enriched binders to biotinylated RBD and the full-length spike protein (Wuhan-1 strain) over 4 rounds of phage panning. The enriched DARPin library pool from the final round was cloned into an expression vector with 6×His and Myc tags at the N-terminus and transformed into E. coli BL21 (DE3) cells. Three hundred colonies were picked and grown in 96-well plates, and the cell lysate was subjected to ELISA-based screens. Eleven unique clones bound strongly to both the RBD and the full-length (fl-) spike protein and were affinity purified. A competitive ELISA was used to identify DARPin molecules that could inhibit binding between hACE2 and spike protein yielding two unique clones: SR16 and SR22. SDS-PAGE analysis of these proteins revealed a homogenous band for SR22 but an additional band for SR16. The N-terminal 6×His tag in SR16 was moved to the C-terminus to give rise to SR16m.

Since the SARS-COV-2 spike protein adopts a trimeric structure, we trimerized both SR16m and SR22 through fusion to T4 foldon (Guthe, S. et al., (2004), J Mol Biol 337, 905-915, doi: 10.1016/j.jmb.2004.02.020) a small trimeric protein, with a flexible (GGGGSLQ; SEQ ID NO: 1)×2 linker to form FSR16m and FSR22 (FIG. 1). Subsequently, using lentiviruses pseudotyped with the Wuhan-1 spike protein, we determined the neutralization activity of SR16m and SR22. Monomeric SR16m and SR22 showed weak activity toward the historical Wuhan-1 virus with IC₅₀ values of 11 μM and 0.7 μM, respectively, which were enhanced 35,000- and 3,800-fold upon trimerization (Table 1), underscoring a strong avidity effect of the trimeric configuration. Despite the use of the Wuhan-1 spike protein during DARPin engineering, both FSR16m and FSR22 exhibited greater neutralization activity toward viruses pseudotyped with spike proteins from newly emerged VOCs. As an example, the IC₅₀ values of FSR16m and FSR22 toward the Omicron variant are 8.5 pM and 6.2 pM, respectively, which are 35- and 300-fold more potent than toward viruses displaying Wuhan-1 spike protein (331 pM and 1.8 nM, respectively).

All monomeric and trimeric DARPin proteins were efficiently expressed in E. coli with yields of ˜200 mg/mL and were easily purified by one-step affinity chromatography. The presence of surface Cys residues (FIG. 1) did not impact the tertiary structure of these proteins. Monomeric DARPin SR16m migrated slightly faster than SR22 on both SDS-PAGE gel and on size exclusion chromatography, while their respective trimers had nearly identical size. FSR16m was highly homogenous in solution, whereas a small percentage of FSR22 possibly aggregated in non-reducing conditions.

FSR16m and FSR22 bind diverse RBD variants with high affinity. FSR16m exhibited superior binding affinity than FSR22 against a panel of spike proteins from VOC and VOI (Table 2) in the Bio-Layer Interferometry (BLI) binding assay. Due to the negligible dissociation rate, the KDapp values of FSR16m for the RBD of Wuhan-1 and 6 viral variants were below the detection limit (KDapp<1 pM). In the case of Omicron RBD, a faster dissociation rate was observed, resulting in a KDapp of 3.65 nM. This result contrasted with our pseudotyped neutralization assay in which the FSR16m exhibited 35-fold improved IC₅₀ values against the Omicron variant compared to Wuhan-1 virus. The difference in the apparent binding affinity may be due to a difference in the tertiary conformation of the protein in solution and on the virus. The binding study used dimeric Fc-tagged RBD whereas the trimeric spike protein was displayed on the pseudotyped lentiviruses. FSR22 maintained a similar binding affinity toward all tested RBDs with a slight increase in affinity to the RBD of Omicron (KDapp=2.63 nM) than Wuhan-1 (KDapp=12.3 nM). Overall, a faster dissociation rate was observed for FSR22 than FSR16m.

To assess the breadth of FSR16m and FSR22 interaction with other SARS-COV-2 VOC and VOI, we determined their binding activity for a panel of 24 RBD mutants by ELISA. Remarkably, FSR16m maintained nanomolar affinity toward all variants except for ones with K417E and F486V/S substitutions (Table 2). Mutation K417E was predicted to escape antibody neutralization from a pseudotyping study. However, spike protein with K417E mutation also exhibits significantly reduced binding affinity for hACE2 (17.8% of Wuhan-1 control) due to the abrogation of a critical salt bridge interaction between the positively-charged K417 in the spike protein and the negatively-charged Asp in hACE2. K417N/T substitutions are present in B.1.351 (Beta) and P.1 (Gamma) variants, respectively (Table 2).

The affinity of FSR16m toward the B.1.351 RBD (K417N+E484K+N501Y, EC50 0.43 nM) is similar to the Wuhan-1 RBD (EC50 of 0.59 nM) but slightly weaker than that to variant with only E484K+N501Y (EC50 of 0.23 nM), indicating that the K417N mutation slightly weakens the interaction between FSR16m and the spike protein. Similarly, the mutations F486V and F486S also exhibit decreased hACE2 binding affinity (37% and 57%, respectively, of Wuhan-1 control). Consequently, both K417E and F486V/S mutations might result in reduced viral fitness, precluding their domination in humans. Mutations G476S and G446V are previously found to be resistant to neutralization by antibodies REGN-10933 and REGN-10987, respectively. Despite a greater than 10-fold reduction in EC50 values, FSR16m still maintained nanomolar binding affinity toward both mutations (Table 2).

FSR22 retained high-affinity binding to most of the tested variants with EC50<10 nM, although its binding strength was generally lower than FSR16m. Similar to FSR16m, FSR22 also exhibits weakened affinity toward RBDs harboring mutations K417E, F486V/S, G446V, or G476S. Beyond these, FSR22 had reduced affinity to RBDs containing T478K substitution, pointing to its occupancy of a slightly different binding interface than FSR16m. The ability of FSR16m and FSR22 to bind diverse RBD variants with high affinity reflects a tendency of these DARPins to mimic hACE2 toward which the virus is obligated to maintain a high binding affinity for efficient infection.

### In Vivo Protection by FSR16m.

We next tested the ability of FSR16m and FSR22 to neutralize authentic SARS-CoV-2 in Vero-hACE2-TMPRSS2 cells. Both FSR16m and FSR22 DARPins efficiently inhibited infection of several authentic SARS-COV-2 strains with FSR16m exhibiting >10-fold greater potency (Table 3). The IC₅₀ values of FSR16m against the authentic B.1.351 (Beta), B.1.617.2 (Delta) and B.1.617.2.AY1 viruses were 3.4, 2.2, and 3.2 ng/mL, respectively. These values are similar to that of the Regen-COV antibody cocktail.

The in vivo efficacy of FSR16m was evaluated in mice. Eight-week-old female heterozygous K18-hAce2 C57BL/6J mice were administered 10³ focus-forming units (FFU) of SARS-COV-2 B.1.617.2 on day 0. On day 1 and day 4 post-infection, FSR16m (50 μg/mouse in PBS) was administered via an intranasal route. FSR16m-treated mice had less weight loss and 10-100-fold lower levels of viral RNA in the lung, heart and nasal wash than mice treated with PBS (FIGS. 3B-3D). Thus, FSR16m showed therapeutic efficacy against SARS-COV-02 as an intranasally administered protein.

Cryo-EM Structures of FSR22 and FSR16m in Complex with SARS-COV-2 Spike Revealed that the DARPin Molecules Recognized an Essential Subset of the ACE2-Binding Surface.

To understand pan-neutralizing activity of SARS-COV-2 variants by FSR22 and FSR16m, we determined their cryo-EM structures of FSR22 and FSR16m in complex with SARS-COV-2 S6P spikes. Ab-initio reconstructions of cryo-EM images of the complexes followed by heterogeneous refinements revealed trimeric SARS-COV-2 spikes with a variety of RBD conformations, ranging from all-down to one, two, or three RBD-up conformations—with SR22 or SR16m binding to the up-RBD in all cases. As FSR22 and FSR16m, trimeric forms of three SR22/SR16m linked by a foldon, were the most potently inhibitory, we focused on refinement of three RBD-up conformations of SARS-COV-2 spikes with the FSR22/16m bound on the tip of the RBD in its open conformation. Overall, the electron density of the trimeric spike complexes was well defined except for the tip of RBD and the FSR22/FSR16m due to high heterogeneity in conformations. As a result, we could not delineate the FSR22/FSR16m-RBD interface in atomic detail, even after extensive heterogeneous refinement followed by local refinement. However, the overall fold of the DARPins and the tip of RBDs fit well, revealing FSR22 and FSR16m to bind the “tip” of the RBD in their “RBD-up” conformation. The binding mode of each of the two DARPins to SARS-COV-2 spike was virtually identical. Therefore, we used the FSR22-RBD structure, which we determined at higher resolution than FSR16m, to model FSR16m residues that differ from FSR22, as the overall fold of the two DARPins were nearly the same. The FSR22-bound spike showed that residues Pro45, Ser46, Val78, Trp79, Arg81, Phe89, Asn112, Tyr114, Leul 19, Arg123 and Phe145 of SR22 interacted with spike residues Leu455, Phe456, Glu484, Phe486, Asn487, Tyr489, and Phe490 on RBD. Similarly, residues Leu45, Phe46, Ala78, Phe79, Arg81, Leu89, Tyr112, Val114, Leu119, Leu123 and Phe145 on SR16m contacted spike residues Lys417, Tyr421, Leu455, Phe456, Gln474, Glu484, Gly485, Phe486, Asn487, Tyr489, and Phe490 on RBD. These data explain the reduced affinity of FSR16m and FSR22 for mutations K417E and F486V/S. Of note, the FSR22/FSR16m contacting surface encompassing residues Leu455, Phe456, Ala475, Glu484, Gly485, Phe486, Asn487, Tyr489 and Phe490 coincided with the hACE2-binding surface. Moreover, residues Tyr421, Phe456, Phe486, Asn487 and Tyr489, which make up roughly two-thirds of the entire DARPin-interactive surface, did not overlap with residues that underwent mutations in variants of concern (VOC), such as N501Y in B.1.1.7 (Alpha), K417N, E484K, and N501Y in B.1.351 (Beta) variant, L452R and E484Q in B.1.617.2 (Delta) variant, and K417N, S477N, Q498R, and N501Y in B.1.1.529 (Omicron) variant, suggesting that the surface recognized by FSR22/16m is particularly important for ACE2 binding and changes in these residues may therefore compromise viral infectivity and fitness.

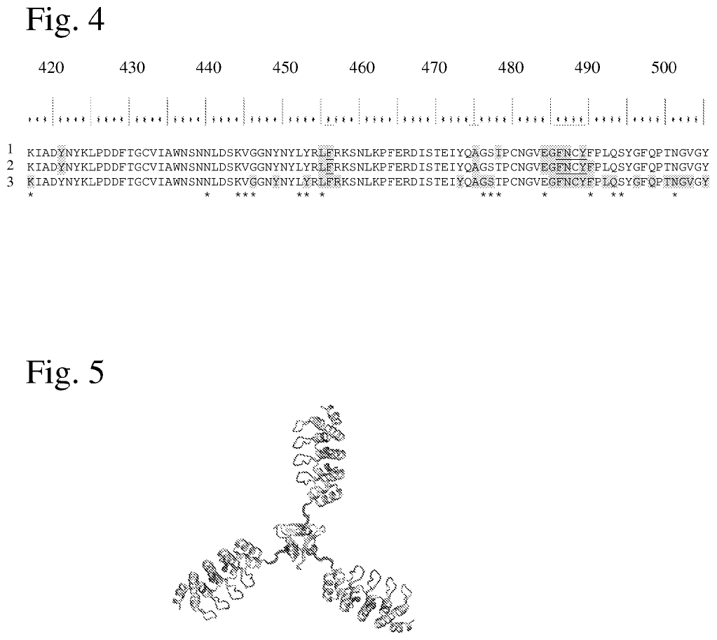

Comparison of the two DARPins RBD structures and the ACE2-RBD structure showed that a monomer of SR22/SR16m and ACE2 binding to RBD sterically clash, as the DARPins and ACE2 recognize overlapping epitopes. However, SR22 and SR16m only buried about 470 Å² of surface area on RBD, whereas ACE2 binding buried about 970 Å² of the surface on RBD, explaining the structural basis for the relatively poor neutralizing ability of SR22/SR16m (monomers) as they could be outcompeted by ACE2 for RBD binding. Conversely, FSR22 and FSR16m, composed of three SR22 and SR16m, respectively, can overcome the lower affinity of monomeric DARPins to RBD, by binding three RBDs in its open conformation. Collectively, the cryo-EM structures of FSR22 and FSR16m-bound SARS-COV-2 structures revealed that FSR22 and FSR16m potently neutralize SARS-COV-2 including VOC by targeting a minimal but essential subset of ACE2-binding surface and taking advantage of avidity gained by their trimeric forms (FIG. 5), which could not be attained by a monomeric SR22 or SR16m.

### CONCLUSIONS

The use of phage panning coupled with functional screening produced engineered DARPins with potent and broad neutralizing activity against SARS-COV-2. Monomeric DARPin SR16m and SR22 exhibited weak viral neutralization potency, but this was enhanced 35,000- and 3,800-fold, respectively, upon trimerization by fusion with T4 foldon. The best DARPin-FSR16m-neutralized authentic SARS-COV-2 B.1.617.2 (Delta) with IC₅₀ of 2.2 ng/mL, similar to currently used therapeutic antibodies. Although the Wuhan-1 spike protein was used for engineering, FSR22 and FSR16m exhibited 35-300-fold increased neutralization potency against viral variants, and potently neutralized pseudotyped viruses displaying Omicron RBD with IC₅₀s of 8.5 pM and 6.2 pM, respectively.

Cryo-EM structural analyses revealed that both FSR22 and FSR16m recognize a distinct subset of the ACE2-binding surface on RBD, particularly residues 421, 456 and 485-489. Shang et al. demonstrated that mutations on the residues 481-489 reduced ACE2 binding substantially (Shang, J. et al. (2020) Nature 581, 221-224, doi: 10.1038/s41586-020-2179-y), and Starr et al. reported that mutations in residues 421, 475 and 487 reduced RBD expression and mutations in residues 421, 456, 475, 486, 487 and 489 reduced ACE2 binding affinity (Starr, T. N. et al. (2020) Cell 182, 1295-1310.e1220, suggesting that the precise targeting of essential ACE2 interacting residues on RBD by FSR22 and FSR16m may explains their pan-neutralization of naturally derived SARS-COV-2 and that the two DARPins may be effective in preventing infection from any newly emerging variants.

The ability of these DARPins to exhibit increased neutralization potency toward newly evolved viral variants contrasts with human-derived anti-SARS-COV2 monoclonal antibodies which have lost neutralization potency toward viral variants. We speculate that multiple reasons may be explain this phenomenon. (a) Naturally derived viral variants are selected in part by their ability to evade the neutralization by circulating human antibodies, including many developed for human therapy. This contrasts with in vitro engineered DARPins whose potency is independent of the virus evolution history. (b) The dimeric structure of an antibody limits a maximum of two spike protein within a spike trimer to be simultaneously engaged by a single antibody, necessitating a high affinity between the antibody and the spike protein and a close match of the binding interface. Consequently, small changes at or near the binding interface could disrupt antibody-spike protein interactions that reduce antibody potency. In contrast, due to its smaller size, a trimer DARPin can engage all three spike proteins in a spike trimer concurrently. The strong avidity effect from the trimer interaction can compensate for the poor binding affinities of the monomers and enable the trimers to tolerate multiple mutations at or near the binding interface without compromising potency. (c) DARPin SR16 and SR22 were selected based on their ability to compete with hACE2 for binding to the spike protein rather than their ability to inhibit viral infection. An effective way to inhibit ACE2 binding to spike is to occupy the critical interface for hACE2 binding, and indeed both DARPins occupy key epitopes within the hACE2 binding interface. As emerging natural VOC tend to exhibit higher infectivity and ACE2 binding affinity, these variants also become more vulnerable to binding and neutralization by our hACE2-mimicking DARPin molecules.

The upper airway epithelium is a postulated first site of infection by SARS-COV-2 due to its robust expression of hACE2. Infected upper airway epithelium cells release progeny viruses, which lead to infection of other organs including the lung. Antibody therapeutics have been an effective weapon for treating COVID-19, with several virus-neutralizing IgG antibodies approved for emergency use by the FDA. However, antibodies suffer from several limitations as therapeutics for a global pandemic: (a) antibody production requires sophisticated mammalian cell culture and is expensive with limited global manufacturing capacity; (b) IgG antibody requires subcutaneous or intravenous routes of administration that may be inconvenient for patients and care providers; (c) intravenously and subcutaneously administered antibodies have poor access to mucosal compartments with an estimated 50- to 100-fold lower antibody levels than in blood, necessitating a high therapeutic dose (up to 8 grams per patient); and (d) many current COVID antibody therapeutics exhibit narrow neutralization spectrum and often require a cocktail of at least 2 different antibodies to maintain efficacy toward newly emerged SARS-COV-2 variants. The most recent Omicron variant was resistant to bamlanivimab, etesevimab and REGEN-COV (casirivimab and imdevimab).

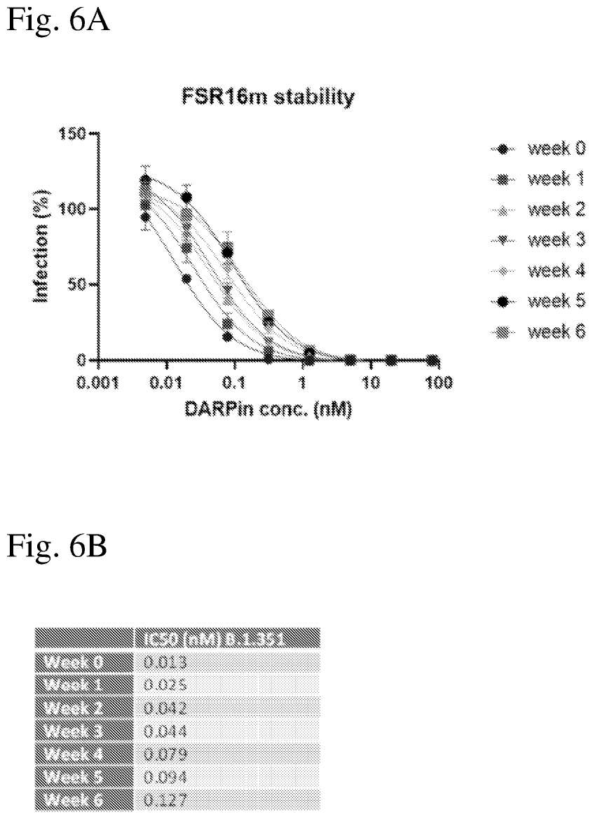

Through the use of stringent K18-hACE2 mouse model of SARS-COV-2 pathogenesis, we showed that intranasal administration of FSR16m on days 1 and 4 post infection effectively protected mice from infection and weight loss. Several protein-based antivirals are currently under development against COVID-19. However, unlike FSR16m, which potently neutralized all tested VOCs, many of these antibodies suffer narrow spectrum of neutralization. FSR16m can also be very efficiently expressed in E. coli. (>200 mg per liter of shaker flask culture), enabling cost-effective production in large-scale. In addition, FSR16m exhibits remarkable stability with <10-fold loss in activity after storage at room temperature for 6 weeks, consistent with previously reported DARPin storage stability and making the molecule a promising therapeutic candidate for intranasal delivery (See FIGS. 6A & 6B).

Previously, two highly potent anti-SARS-COV-2 hetero-trimeric DARPin molecules were reported. One of these DARPins, MM-DC (MP0420, ensovibep), binds three distinct epitopes in RBD, whereas the other, MR-DC (MP0423), targets one epitope on RBD, one on the S1 N-terminal domain, and another on the S2 domain of the spike protein. Proline-threonine-rich polypeptide linkers with designed lengths were used to connect the different DARPin molecules. Both MM-DC and MR-DC exhibited picomolar IC₅₀ values against spike protein pseudotyped VSV particles and were 10-50-fold superior to their constituent monomer DARPins. This potency enhancement is in contrast to both FSR16m and FSR22 whose IC₅₀ values improved 35,000- and 3,800-fold, respectively, upon homo-trimerization. When administered intraperitoneally at 10 mg/kg, MR-DC reduced the viral load in nasal turbinates and lung by ˜10 and ˜100-fold, respectively. A similar level of viral load reduction in these tissues was achieved by intranasally-administered FSR16m at 2.5 mg/kg per animal. Despite targeting three distinct regions on the spike protein, MR-DC (MP0423) lost efficacy against B.1.351 (Beta), B.1.1.7 (Alpha) and P.1. MM-DC (MP0420) retained efficacy against all these variants and its IC₅₀ toward authentic B.1.351 (7.5 ng/ml) is similar to that of FSR16m (3.4 ng/mL). Both MM-DC and MR-DC are currently in clinical development. Thus, DARPins reported previously and those reported here show dramatic differences in their recognition of variants of concern.

Although DARPin molecules have been found to exhibit low immunogenicity, the trimerization domain T4 foldon is of non-human origin and may be immunogenic in humans if administered intravenously. However, since systematic absorption of intranasally administered FSR16m is anticipated to be low due to its large size, FSR16m should remain effective during the narrow treatment window. Nevertheless, efforts to replace T4 foldon with an equivalent trimeric protein of human origin are contemplated and within the scope of the present disclosure.

1. A SARS-COV-2 neutralizing protein, said protein comprising a designed ankyrin repeat protein (DARPin) having at least 95% sequence identity to SEQ ID NO: 12.

2. The SARS-COV-2 neutralizing protein of claim 1 wherein said neutralizing protein further comprises a trimeric foldon peptide covalently linked to said DARPin.

3. The SARS-COV-2 neutralizing protein of claim 2 wherein said DARPin comprises the amino acid sequence of SEQ ID NO: 12.

4. The SARS-COV-2 neutralizing protein of claim 3 wherein said trimeric foldon peptide comprises the sequence of AYVRKDGEWVLL (SEQ ID NO: 14).

5. The SARS-COV-2 neutralizing protein of claim 1 wherein said DARPin comprises an amino acid sequence selected from the group consisting of SEQ ID NO: 5, SEQ ID NO: 6, SEQ ID NO: 7, SEQ ID NO: 8 and SEQ ID NO: 9.

6. The SARS-COV-2 neutralizing protein of claim 5 comprising a sequence of SEQ ID NO: 13 covalently linked to said protein.

7. The SARS-COV-2 neutralizing protein of claim 1 wherein said neutralizing protein comprises an amino acid sequence having at least 95% sequence identity with SEQ ID NO: 10.

8. A pharmaceutical composition comprising a SARS-COV-2 neutralizing protein of claim 1 and a pharmaceutically acceptable carrier.

9. The pharmaceutical composition of claim 8 wherein the composition is formulated for intranasal delivery.

10. A method of enhancing the specific binding avidity of a DARPin for its target protein, said method comprising covalently linking the DARPin to a peptide comprising the sequence of AYVRKDGEWVLL (SEQ ID NO: 14).

11. The method of claim 10 wherein the DARPin is covalently linked to SEQ ID NO: 13.

12. A method of treating a patient exposed to a SARS-COV-2 virus, said method comprising the administration of a pharmaceutical composition comprising a SARS-COV-2 neutralizing protein of claim 1.

13. The method of claim 12 wherein the patient has a SARS-CoV-2 infection.

14. The method of claim 12 or 13 wherein the patient is administered a pharmaceutical composition comprising a SARS-COV-2 neutralizing protein of claim 1 within 7 days after appearance of symptoms of infection or a positive test for infection.

15. The method of claim 12 wherein the method comprises the administration of a pharmaceutical composition comprising a SARS-COV-2 neutralizing protein having at least 95% sequence identity with SEQ ID NO: 10 or SEQ ID NO: 11.

16. The method of claim 12 is provided wherein the method comprises the administration of a pharmaceutical composition comprising two SARS-COV-2 neutralizing proteins, selected from the group consisting of sequence SEQ ID NO: 10 and SEQ ID NO: 11.

17. The method of claim 16 wherein the two administered SARS-COV-2 neutralizing proteins are administered sequentially within 24 or 48 hours of each other.

18. The method of claim 16 wherein the two administered SARS-COV-2 neutralizing proteins are administered simultaneously.

19. The method of claim 12 wherein the pharmaceutical composition is administered intranasally.

20. The SARS-COV-2 neutralizing protein of claim 1 wherein said neutralizing protein comprises an amino acid sequence having at least 95% sequence identity with SEQ ID NO: 10.


SEQUENCE LISTING (15 sequences)

SEQ ID NO: 1 - protein, 7 aa, synthetic construct
  REGION 1..7: note = peptide linker
GGGGSLQ

SEQ ID NO: 2 - DNA, 20 bp, synthetic construct
  misc_feature 1..20: note = forward primer
  source 1..20: mol_type = other DNA
atgctgcaatcgtgctacaa

SEQ ID NO: 3 - DNA, 17 bp, synthetic construct
  misc_feature 1..17: note = reverse primer
  source 1..17: mol_type = other DNA
gactgccgcctctgctc

SEQ ID NO: 4 - DNA, 20 bp, synthetic construct
  misc_feature 1..20: note = Probe
  source 1..20: mol_type = other DNA
tcaaggaacaacattgccaa

SEQ ID NO: 5 - protein, 159 aa, synthetic construct
GSDLGKKLLEAARAGQDDEVRILMANGADVNALDLFGVTPLHLAAERGHLEIVEVLLKNGADVNAGDAFGRTPLHLAALGGHLEIVEVLLKNGADVNACDLYGVTPLHLAAGLGHLEIVEVLLKNGADVNAQDKFGKTAFDISIDNGNEDLAEILQSSS

SEQ ID NO: 6 - protein, 159 aa, synthetic construct
GSDLGKKLLEAARAGQDDEVRILMANGADVNACDPSGITPLHLAADKGHLEIVEVLLKYGADVNAMDVWGRTPLHLAAFTGHLEIVEVLLKYGADVNACDLNGYTPLHLAAGRGHLEIVEVLLKNGAGVNAQDKFGKTAFDISIDNGNEDLAEILQSSS

SEQ ID NO: 7 - protein, 190 aa, synthetic construct
MGSSHHHHHHSSGLVPRGSHMEQKLISEEDLGSDLGKKLLEAARAGQDDEVRILMANGADVNALDLFGVTPLHLAAERGHLEIVEVLLKNGADVNAGDAFGRTPLHLAALGGHLEIVEVLLKNGADVNACDLYGVTPLHLAAGLGHLEIVEVLLKNGADVNAQDKFGKTAFDISIDNGNEDLAEILQSSS

SEQ ID NO: 8 - protein, 174 aa, synthetic construct
MGSSDLGKKLLEAARAGQDDEVRILMANGADVNALDLFGVTPLHLAAERGHLEIVEVLLKNGADVNAGDAFGRTPLHLAALGGHLEIVEVLLKNGADVNACDLYGVTPLHLAAGLGHLEIVEVLLKNGADVNAQDKFGKTAFDISIDNGNEDLAEILQSSSKLAAALEHHHHHH

SEQ ID NO: 9 - protein, 190 aa, synthetic construct
MGSSHHHHHHSSGLVPRGSHMEQKLISEEDLGSDLGKKLLEAARAGQDDEVRILMANGADVNACDPSGITPLHLAADKGHLEIVEVLLKYGADVNAMDVWGRTPLHLAAFTGHLEIVEVLLKYGADVNACDLNGYTPLHLAAGRGHLEIVEVLLKNGAGVNAQDKFGKTAFDISIDNGNEDLAEILQSSS

SEQ ID NO: 10 - protein, 227 aa, synthetic construct
MGMEQKLISEEDLDGYIPEAPRDGQAYVRKDGEWVLLSTFLGGGGSLQGGGGSLQGSDLGKKLLEAARAGQDDEVRILMANGADVNALDLFGVTPLHLAAERGHLEIVEVLLKNGADVNAGDAFGRTPLHLAALGGHLEIVEVLLKNGADVNACDLYGVTPLHLAAGLGHLEIVEVLLKNGADVNAQDKFGKTAFDISIDNGNEDLAEILQSSSKLAAALEHHHHHH

SEQ ID NO: 11 - protein, 225 aa, synthetic construct
MGSSHHHHHHSSGMEQKLISEEDLDGYIPEAPRDGQAYVRKDGEWVLLSTFLGGGGSLQGGGGSLQGSDLGKKLLEAARAGQDDEVRILMANGADVNACDPSGITPLHLAADKGHLEIVEVLLKYGADVNAMDVWGRTPLHLAAFTGHLEIVEVLLKYGADVNACDLNGYTPLHLAAGRGHLEIVEVLLKNGAGVNAQDKFGKTAFDISIDNGNEDLAEILQSSS

SEQ ID NO: 12 - protein, 159 aa, synthetic construct
  VARIANT 33: note = Xaa can be any naturally occurring amino acid
  VARIANT 35..36: note = Xaa can be any naturally occurring amino acid
  VARIANT 38: note = Xaa can be any naturally occurring amino acid
  VARIANT 46..47: note = Xaa can be any naturally occurring amino acid
  VARIANT 59: note = Xaa can be any naturally occurring amino acid
  VARIANT 66: note = Xaa can be any naturally occurring amino acid
  VARIANT 68..69: note = Xaa can be any naturally occurring amino acid
  VARIANT 79..80: note = Xaa at position 79 and 80 is any amno acid
  VARIANT 92: note = Xaa can be any naturally occurring amino acid
  VARIANT 102: note = Xaa can be any naturally occurring amino acid
  VARIANT 113: note = Xaa can be any naturally occurring amino acid
  VARIANT 128: note = Xaa can be any naturally occurring amino acid
GSDLGKKLLEAARAGQDDEVRILMANGADVNAXDXXGXTPLHLAAXXGHLEIVEVLLKXGADVNAXDXXGRTPLHLAAXXGHLEIVEVLLKXGADVNACDLXGYTPLHLAAGXGHLEIVEVLLKNGAXVNAQDKFGKTAFDISIDNGNEDLAEILQSSS

SEQ ID NO: 13 - protein, 40 aa, Bacteriophage T4
GMEQKLISEEDLDGYIPEAPRDGQAYVRKDGEWVLLSTFL

SEQ ID NO: 14 - protein, 12 aa, Bacteriophage T4
AYVRKDGEWVLL

SEQ ID NO: 15 - protein, 89 aa, SARS-CoV-2
KIADYNYKLPDDFTGCVIAWNSNNLDSKVGGNYNYLYRIFRKSNLKPFERDISTEIYQAGSTPCNGVEGFNCYFPLQSYGFQPTNGVGY